(19) United States
(12) Patent Application Publication
(10) Pub. No.: US 2019/0328385 A1
(43) Pub. Date: Oct. 31, 2019

(54) MEDICAL DEVICE

(71) Applicants: Violeta DOCI, Hamburg (DE); Donata michelle DOCI, Hamburg (DE)

(72) Inventor: VIOLETA DOCI, Hamburg (DE)

(21) Appl. No.: 16/074,831

(22) PCT Filed: Feb. 6, 2017
(86) PCT No.: PCT/EP2017/052570

§ 371 Date: Nov. 2, 2018

(30) Foreign Application Priority Data
Feb. 4, 2016 (DE) 10 2016 101 937.2

(51) Int. Cl. A61B 17/06

(52) U.S. Cl. CPC A61B 17/06066; A61M 25/0082; A61B 2017/06076

(57) ABSTRACT

The invention relates to a medical device for positioning a thread in tissue. In particular, the invention relates to a medical device (10) comprising a spiral needle (12), wherein the spiral needle (12) has a spiral-shaped needle body (14) which is formed about a central axis (A) and runs along a spiral path and also has a front end (16) and a rear end (18), wherein at least the front end (16) is integrally formed with the needle body (14), wherein the spiral path has an outer tangential plan (Ta), an inner tangential plane (Ti), an upper side (O) and an opposing underside (U), arid wherein the front end (16) runs in the direction of the spiral path of the needle body (14), and the front end (16) has a tip (22) and a thread-receiving means (24) arranged behind same.

### FIELD OF THE INVENTION.

The present invention relates to a medical device that can be inserted into tissue with the sutures. In this sense, it is primarily a surgical device. The device can find application in the healing-medical field, but also in commercial areas.

### BACKGROUND OF THE INVENTION

The international patent application WO 01/30245 A1 discloses a surgical instrument can be placed with the sutures. This instrument consists essentially of two tubular elements, said first tube defining a central axis and the second tube is guided as a helix about this axis. The end of the second tube is tapered. The instrument is basically serve to place a suture at which the yarn is guided spirally. For this, the helix-shaped tube along the intended yarn path is passed through tissue. The thread can then be placed in the advancing phase or in the withdrawal phase in the tissue.

The U.S. patent application US 2012/0136200 discloses another surgical instrument. This is specifically designed for surgical procedures on the heart. The apparatus includes a handle a meddle puncturing needle is held at the front end. To the puncturine needle around a helix-shaped needle is arranged. The puncture needle can be inserted into a heart wall. In order specified by the puncturing needle point, a thread can be placed by means of the helix-shaped needle.

The two disclosed in these patent applications are instruments designed far specific forms of placement surgical sutures. However, the sutures are essentially used in the conventional way laying a surgical suture, so the at least temporary connection of tissue. Both instruments can be adopted only if at least two needles are used simultaneously and correspondingly also cut two needles tissue. The present invention seeks to provide a novel medical device. This is intended for gentle and controlled Put surgical sutures, which need not necessarily meant the connection of two fabric pieces. Tissue is intended to be protected as possible. This object is achieved by a medical device according to Claim 1

### MORE DETAILED DESCRIPTION

The medical device can be used by a physician or by other medical and non-medical personnel. It can be used for the treatment, in particular to operations, in humans or in animals. The application in the medical or healing area, but also a merely commercial application be considered. In particular, the medical device is suitable for placing threads in a fabric. For example, threads can be placed so that they fill tissue. In particular, the relining of skin wrinkles can be considered. Therefore, the medical device can advantageously be used in cosmetic surgery. This medical device is also adapted to guide a yarn so by tissue in that the fabric, in particular its outer appearance, is tightened by the thread. Here, too, an application in the context of cosmetic surgery can be considered.

The medical device is intended in particular comprise a helical needle, a needle at least partially has a spiral shape. The spiral needle has a spiral needle body, which is formed around a central axis. Around this axis, the needle body extending along a spiral path. The needle body has a front end and a rear end. In this case, the front end is formed at least integrally with the needle body. The integral formation can be obtained by (possibly detachable, but substantially) fixed connections of a part that forms the front end, be effected with the needle body. The transition between the needle body and the part forming the front end, then should be substantially smooth and without joints. Suitably, the front end is generally integrally formed with the needle body. The rear end may be integrally formed with the needle body expediently likewise, again with integral formation is particularly useful. The front end extends in the direction of the spiral path of the needle body, that is, the history of the needle body continues to the front end, without kink is formed. The front end has a tip, and a thread take-up arranged behind, wherein the top, or more precisely the end point or apex of the tip, the outer end of the front end labeled.

It is particularly advantageous if the helical needle body and usually also the front end and the rear end are solidly constructed. That is, these parts are not made of a hollow body and, accordingly, have no other lumen or cavity.

The thread take-up can be arranged in various forms, functional is an opening. This opening can be round or oval. The opening may be a slot. Along the spiral path, the needle body has an outer surface and an inner tangent tangent space. The Tagentenflächen be defined by two axes. One of these axis is a tangent to the outer side or inner side of the spiral path. The other axis is parallel to the central axis (A). The inside tangent surface is closer to the central axis as the outer surface tangent. The spiral needle body further comprises a top and a bottom, these opposite. The upper surface is oriented generally in the direction of the rear end and, optionally, a handle piece.

It is expedient if the opening of the top of the needle body to the bottom of the needle body is running.

It is advantageous when the front end between the outer tangent surface and the inner surface is tapered tangent is. Thus, when observing a cross section perpendicular to these surfaces (which then also extends substantially parallel to the top and bottom of the needle body), so in this cross section, a peak is visible. This tip is advantageously triangular. Suitably an isosceles triangle, is useful in any case, if the end point or apex of the tip is arranged centrally between the outer surface and the inner tangent line tangent surface.

It is also expedient if the front end between the top and the bottom of the needle body is tapered. In this way also results in a cross-sectional area perpendicular to the top and to the bottom (which is to say parallel to the outer surface tangent and the inner surface is tangent) peak. It is expedient for the end point or the peak of the peak at the top is closer than at the bottom. It is particularly useful when the climax is entirely up to the top, that is the point in the extension of the course of the spiral path is.

It has been shown that this type of rate training in the use of the medical device caused particularly low tissue damage and allows readily controllable and precise penetration in the tissue. The surgeon finds it even a little resistance.

It is advantageous if the rear end of the needle body is straight. The rear end therefore does not run along a spiral path, and may extend parallel to the central axis substantially. It is particularly advantageous if the rear end extends to the central axis. This allows forces, especially torques to convey particularly well over the rear end. It is advantageous when the rear end is connected to a handle piece, the medical device on such a handle can be run well and it can easily higher forces, especially torques are applied.

The thread take-up may conveniently have the form of a hole, and indeed has substantially the shape of a round or an oval hole, it is particularly advantageous when a thread take-up comprises a hole two holes or three holes.

Alternatively or additionally, the thread take-up may also be designed as a slot. Conveniently, a substantially straight slot, that is, a slot with a rectangular cross-section. When an elongated rectangular shape is formed, it may also be advantageous to provide a bulge at at least one short ends of such a rectangle, for example, a circular bulge. Such protrusions may also be formed on both opposite shod ends. A suitable slot may also have an oval shape in cross section.

It may be expedient, when the leading end of the medical device having a bulbous portion. Such a bulbous portion can be provided in particular in the amount of the thread take-up. Along the bulbous portion, the diameter of the needle body is increased (along a circular disc measured, which is perpendicular to the spiral track).

Useful is also a medical device, which already has a surgical thread. This thread is expediently already passed through the thread take-up. It is particularly useful if such device is supplied with the thread in sterile packaging. The device can comprise a thread or also more than one thread, for example two, three or four threads.

In the present invention, a set of medical devices is useful, comprising two, three or more devices. These devices can be made with or without threads available. The devices can be configured similarly or differently. For example it is useful to combine a medical device with a left-rotating helical needle and a medical device with a right-rotating helical needle. In this way, a set can be provided with two complementary medical devices.

It is also useful to provide medical devices with different diameter of the needle body available. Also, the length of the device along the central axis A car be varied. Further, the diameter of the needle body can be varied about the central axis. Also medical equipment from different materials, for example with different material hardness, are used. As a rule, surgical steel is used, but there are also other materials for the production of the medical device conceivable, for example plastics. All of these materials are available in different material hardness.

It may be expedient also an apparatus in which the spiral pitch changes along the central axis. This means that a 360-degree rotation of the spiral about the central axis on variable lengths (measured from the central axis) occurs.

Devices with these variations are individually useful for certain applications, but they can also be useful in a set of equipment combined. Further features, as well as advantages of the invention will become apparent from the following drawings and the accompanying description. In the Figures and in the accompanying descriptions features of the invention in combination are described. However, these features may be included in other combinations of an inventive subject matter. So each feature disclosed is also to be regarded as revealed in a technically sensible combinations with other features. The images are partially slightly simplified and schematic.

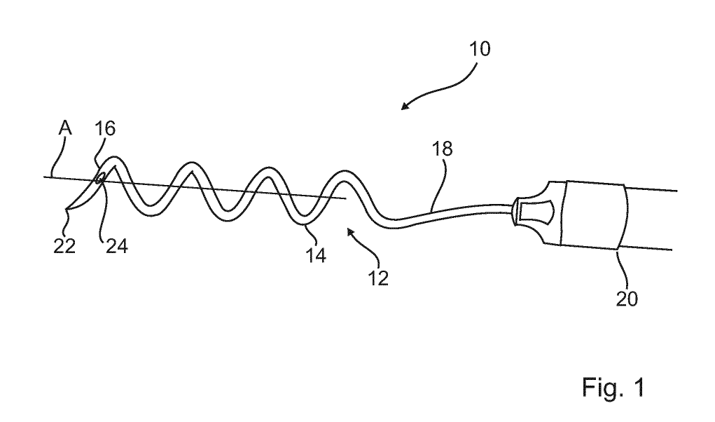

FIG. 1 shows an overall perspective view of a medical device according to the invention.

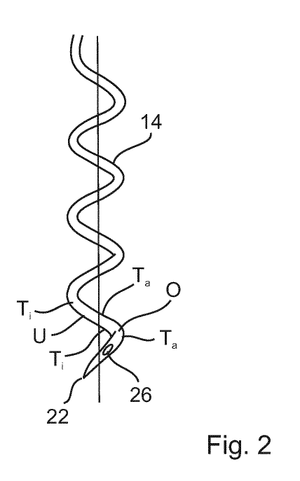

FIG. 2 shows a perspective view of the needle body of the Device.

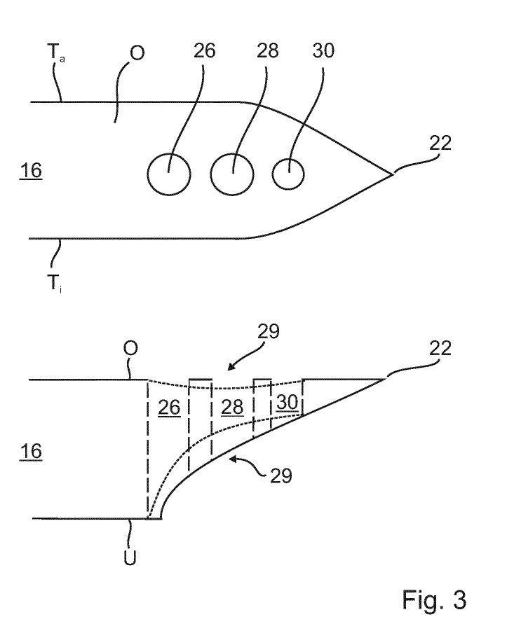

FIG. 3 shows in two cross-sectional views of the front end of Needle body of a device according to the invention.

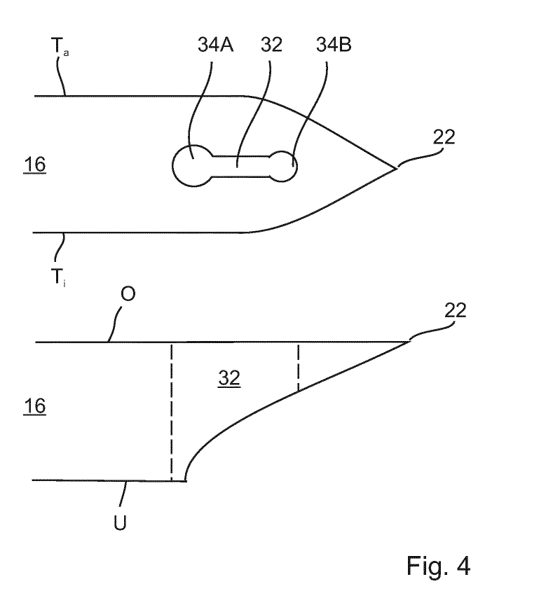

FIG. 4 shows, in two cross-sectional views of the front end other medical apparatus according to the invention.

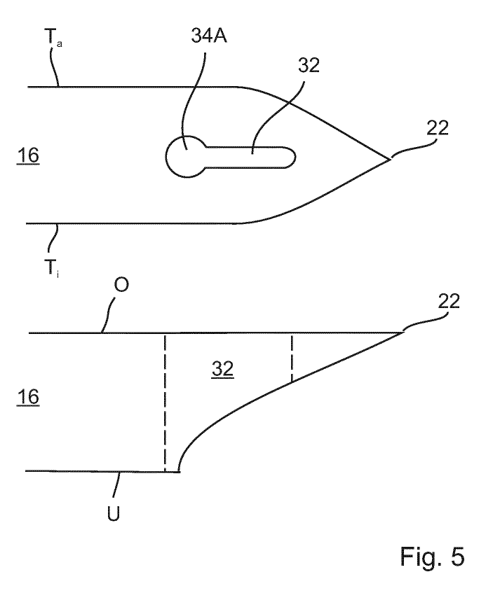

FIG. 5 shows in two cross-sectional views of the front end yet another medical device according to the invention

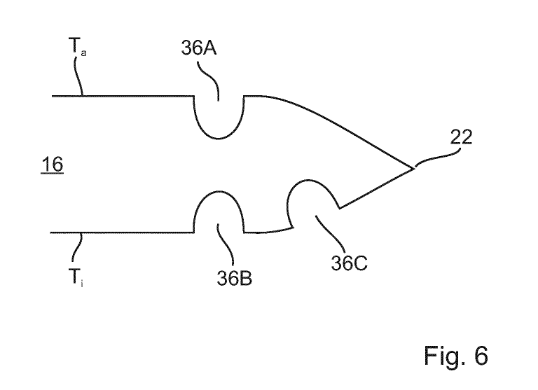

FIG. 6 is a cross-sectional view of the front end of yet another medical device.

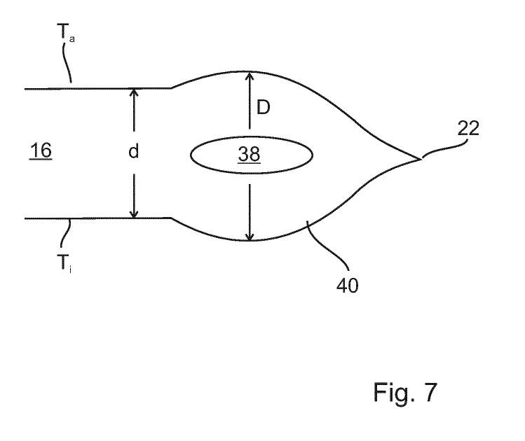

FIG. 7 shows in a corresponding cross-sectional view of the front end of yet another medical device.

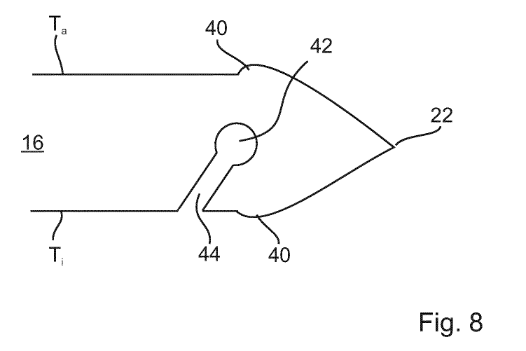

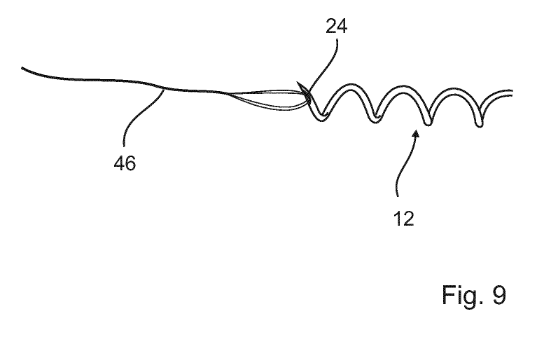

FIG. 8 shows in a corresponding cross-sectional view of the front end of yet another medical device. FIG. 9 shows a perspective view of the needle body of a device according to the invention together with a surgical thread.

FIG. 1 shows a perspective view of a medical apparatus 10 of the present invention. The device has a spiral needle 1 second. This spiral needle comprising a spirally shaped in needle body 14 which extends from a front end to a rear end 16 18. The spiral needle body extending along a spiral, which extends around a central axis A. The rear end 18 is connected to a handle 20th At the front end 16, the medical device 10 to a peak 22nd Also in the area of the front end, but behind the tip 22, a thread take-up 24 is located.

FIG. 2 shows in an enlarged perspective view of the course of the spiral needle body 14. The coil winding, along a spiral path from the rear end (not shown) in the direction towards the tip 22. Behind the tip 22 of the thread take-up is provided which is here in the form of has a single hole 26th Along the spiral path can be theoretically unlimited number of discs think as cross sections through the needle body, an outer surface and an inner tangent surface and a top and a bottom can be defined for each of these slices. The outer surface is tangent by an outer tangent on the disc (and perpendicular to the disk surface) and defined by the central axis. The inner surface tangent is in a corresponding manner by the inner tangent to the disk (more precisely to a disc lying in the ring, whose radial thickness is determined by the needle body) defined and the central axis. About 90 degrees offset regions of the needle body can be described as top side or underside of the needle. Through the respective marks in FIG. 2, Tₐ for the outer tangent area, Ti for the inner tangent area O for top, U for bottom, the respective surfaces easily recognizable (although the view of the underside U in this illustration is not possible). Analogoesly, they are set for a needle slightly different form. FIG. 3 shows in two cross-sectional views of the front end 16 of a needle body of the invention. The front end 16 is limited by a peak 22nd For orientation purposes are a tangent outer surface, an inner surface tangent and the upper side as well as also shown the lower side (in the other cross sectional view). The thread take-up is provided in the form of three holes, namely a first hole 26, a second hole 28 and a third hole 30th

clearly visible as in the second cross-sectional view, these holes connect the top of the needle body with the bottom of the needle body. In general, it is desirable in the present invention, when the front end has an indentation 29 approximately in the range of the thread take-up. This facilitates the management of the tip and protects tissues. The edge portions of the corresponding indentation are marked in the cross sectional view by dashed lines. Along these lines may extend corrugations that limit the recess side.

Although this additional feature is shown only in connection with FIG. 3, it is useful readily with the other embodiments illustrated. In the two cross-sectional views of the tip shape is clearly visible. Between the outer tangent surface and the inner surface tangent to the front end of the climax comes to a head. This is midway between the outer tangent surface and the inner surface tangent. The front end 16 also tapers between the top and bottom U O to. However, this peak is not centered between the two sides, but rather entirely on the side of the top. FIG. 4 shows, in two cross-sectional views, which those of FIG. 3 correspond to a different embodiment of the medical device according to the invention. This has a different design of the front end 16 The to shape of the tip 22 is, however, also be selected as in the device of FIG. 3. Instead of a plurality of holes is provided, however in this apparatus, a slot 32. This slot has substantially, as clearly visible in the upper cross-sectional view, in cross-section the shape of an elongated rectangle. However, hole-like bulging 34A and 34B are provided on the narrow ends of the rectangle. These facilitate receiving a suture.

FIG. 5 shows in two cross-sectional views corresponding to those of FIG. 3, a still other embodiment of a medical device according to the invention. In this apparatus, a slit is again provided 32nd The slot also has in cross section the shape of an elongated rectangle. However, it has a hole-like bulge 34A only at a narrow end of the rectangle. This bulge facilitates the absorption of a thread, which is then performed very safe at the narrower end of the slot.

FIG. 6 shows the front end 16 of another medical device according to the invention. This has no holes, but rather a plurality of, grooves, namely groove 36A, opposite groove 36B, and even closer to the tip 22 through a third groove 36C. Even in these grooves a surgical thread can be firmly anchored.

FIG. 7 shows another embodiment of a medical device according to the invention. In this case, an oval slot 38 is provided for the thread take-up. Further, the front end 16 of the needle body to a bulbous region. In this area, the diameter of the needle body is measured widened perpendicular to the spiral path. Accordingly, the diameter d is given before the bulging portion 14, whereas it is clear in the D bulbous region of the larger diameters.

FIG. 8 shows a cross-sectional view of still another embodiment of a medical device according to the invention. In this case, a hole 42 is received at the front end of the tip behind the 22nd However In this has the thread does not threaded, since the hole is connected through the notch 44 with the Innentangentialfläche of the needle body. Again, a bulbous portion 40 is provided in the vicinity of the hole.

FIG. 9 shows the spiral needle of a medical device according to the invention, which carries in its thread take-up 24 a surgical thread 46th It is expedient that an approximately central portion of the surgical thread is taken up from the thread take-up 24 46th Both ends of the suture can then be performed side by side or even verzwirbelt. In this way, the surgical thread has 46 a secure fit in the thread take up 24 in addition it is possible to double the thread thickness, namely to simultaneously introduce two ends of the thread in the fabric.

Overall, it can be seen, as can make themselves available by the present invention a useful medical device, which is particularly suitable for the described above procedures.

### LIST OF REFERENCES

The medical device 10

12 spiral needle

14 needle body

16 Front end

18 Rear end

20 handle piece

22 tip

24 thread take

26 hole

28 holes

29 recess

30 hole

32 slot

34 (hole-shaped) recess

36 groove

38 oval slot

49 belly

42 hole

44 notch/Implementation

46. A surgical filament

A central axis

Ta outer surface tangent

Ti inner surface tangent

O top

& bottom

1. The medical device (10) comprising a helical needle (12), the helical needle (12) has a spiral needle body (14) having a central axis (A) is formed and extends along a spiral path and also a front end (16 has) and a rear end (18), wherein at least the front end (16) is integral with the needle body (14) is formed, wherein the Spiral path an outer surface tangent (Tₐ) a Inside tangent surface (˜Π), an upper side (O) and one of these opposite underside (U) and wherein the front end (16) extends in the direction of the spiral path of the needle body (14) and the front end (16) a tip (22) and arranged behind thread take-up (24).

2. The medical device (10) according to the preceding claim, wherein said needle body (14) is solid.

3. The medical device (10) according to one of the preceding claims, wherein the thread take-up (24) has an opening (26, 28, 30, 32. 38, 42) and the opening (26, 28, 30, 32, 38, 42) of the top side runs (O) to the underside (U).

4. The medical device (10) according to one of the preceding claims, wherein the front end (16) between the outer surface tangent (Tₐ) and the inner surface tangent (Ti) is tapered.

5. The medical device (10) according to the preceding claim, wherein the center between the outer surface tangent (Tₐ) and the Inside tangent surface (Ti) formed a peak point.

6. The medical device (10) according to one of the preceding claims, wherein the front end (16) between the upper side (O) and the underside (U) is tapered.

7. The medical device (10) according to the preceding claim, being farmed between the upper side (O) and the underside (U), a peak point which is doser to the upper side (O) than on the underside (U).

8. The medical device (10) according to one of the preceding claims, wherein the rear end (18) is straight.

9. The medical device (10) according to the preceding claim, wherein the rear end (18) extends at least partially on the central axis (A).

10. The medical device (10) according to one of the preceding claims, wherein the rear end (18) is connected to a handle (20).

11. The medical device (10), wherein the thread take-up (24) comprises between one and three holes.

12. The medical device (10) according to any one of the preceding claims, wherein the thread take-up (24) has a slot (32, 38).

13. The medical device (10) according to one of the preceding claims, wherein the front end (16) is widened in the area of bulbous thread take-up (24).

14. The medical device (10) according to one of the preceding claims, wherein the front end (16) in the region of the thread take-up (24) a recess (29) is provided.

15. The medical device (10), wherein the thread take-up (24) delivers a surgical thread (44).
